(19) United States
(12) Patent Application Publication
(10) Pub. No.: US 2004/0241186 A1
(43) Pub. Date: Dec. 2, 2004

(54) Aromatherapy formulations and treatment system

(76) Inventor: Wentsan G. Huang, Flushing, NY (US)

Correspondence Address: Donald W. Meeker, Patent Agent, 924 East Ocean Front #E, Newport Beach, CA, 92661, US

(21) Appl. No.: 10/446,387

(22) Filed: May 28, 2003

(51) Int. Cl. A61K 35/78

(52) U.S. Cl. 424/195.18; 424/745; 424/769

(57) ABSTRACT

An aromatherapy treatment system that utilizes a sprayable solution of essential oils that are mixed with distilled water. The solution is applied by means of spraying the desired amount around or on the body of the person being treated, to produce healthful effects. The solution is applied to the seven master chakras and/or the aura of the person being treated, and may be used in conjunction with meditation. A special combination of essential oils is related for treatment each of the seven master chakras, and also for the aura.

### BACKGROUND OF THE INVENTION

1. Field of the Invention

The present invention is directed to improvements in the treatment of persons by aromatherapy, and in particular to improved essential oil mixture formulations and an improved dispersing agent type and concentration.

2. Description of the Prior Art

Aromatherapy in one form or another has been around and has been practiced in one form or another since at least the dawn of civilization. One school of thought traces aromatherapy to ancient Egypt. A medical papyri has been found that is believed to date back to around 1550 BC which contains remedies for a number of illnesses and the methods of application are not unlike some of the ones used in aromatherapy today. The Egyptians used a method known as infusion to extract the oils from aromatic plants and incense is an example of one of the earliest ways of using aromatics. Frankincense was burned at sunrise as an offering to the sun god, Ra and myrrh was offered to the moon. The Egyptians used aromatics in embalming to help preserve flesh. The Egyptians also used fragrant oils after bathing either alone or accompanied by a massage. There is a strong possibility that the ancient Chinese civilizations were using some form of aromatics at the same time as the Egyptians. Shen Nung's Herbal book is the oldest surviving medical book in China which is dated about 2700 BC. and contains information on over 300 plants. The ancient Greeks also used aromatic oils both medicinally and cosmetically.

Aromatherapy may be defined as the art, and science, of using oils extracted from aromatic plants to enhance health, beauty and overall wellness of an individual. These oils are known as essential oils. Apart from the physical benefits, it is believed that the essential oils can have subtle effects on the mind and emotions. The essential oils taken from plants and used in aromatherapy are not only substances which gives the plants their scent but are considered by some to be the plant's “life force” i.e., they are essential to the plants' biological process.

The essential oils used in aromatherapy are extracted from flowers; herbs; spices; woods and fibers, usually by distillation, expression and solvent extraction. Essential oils may be very expensive to produce, some more so than others, due to the labor intensive process and the quantity of the plant required to produce the oil. It requires approximately 400 kg of thyme to produce 1 kg of essential oil, 2000 kg of rose petals to make 1 kg of oil, 6 tons of orange blossom to produce 1 kg of neroli, and 4 million jasmine flowers to produce 1 kg of jasmine absolute. Essential oils have also been known as fragrance oils and are used in the fragrance industry, in their natural or synthetic form. Essential oils, contrary to the use of the word “oil” may not be really oily-feeling at all, rather, essential oil largely refers to the volatile component of plant material.

Aromatherapy uses the essential oils in a variety of ways. One way is in a massage. Some people consider the use of the oils in a massage to be the most effective method of using the oils, because the message combines their oils properties with the therapeutic power of touch. The oils are usually not used undiluted, but are frequently combined with an odorless carrier oil, which may include, such oils as grapeseed, sweet almond or peach kernel. The diluted solution may be about 3% essential oil. Some essential oil constituent chemicals may be absorbed through the skin in small amounts but is believed that the amount is of therapeutic value. Essential oils that are drawn into the body by the sense smell are believed to offer psychological and physical benefits; not only does the aroma of the true plant stimulate the brain to trigger a positive effect, but the natural components of the essential oil are drawn into the lungs and can also supply physical benefits. Careful inhalation of the oils can also provide therapeutic benefit as the oil molecules enter the lungs and are absorbed into the bloodstream. If not done correctly and safely, however, the use of essential oils may also have negative consequences, in some circumstances.

The components of the various oils are believed to aid in a variety of health, beauty and hygiene conditions. Essential oils can supply other benefits as well. Some oils, for instance, act as a natural repellent and pesticide. Essential oils may be blended together to provide an especially pleasing fragrance and are also used to aid in providing a specific health benefit. A synergistic essential oil blend of the correct oils in proper proportions is considered to be greater in total benefit than the individual oils working independently.

One aspect of the application of essential oils and aromatherapy relates to treatment of the body's Chakras. The word chakra is a Sanskrit word meaning wheel or disk. Described as a wheel-like spinning vortex, a chakra is a point of intersection between various planes. The seven major centers along the spinal column have the greatest significance and the highest number of correlations in the treatment of chakras. These seven centers are thought of as the master chakras. Chakras are also called lotuses, symbolizing the unfolding of flower petals that metaphorically describe the opening of a chakra. Like flowers, chakras are believed to be opened or closed, dying or budding, depending on the consciousness within. Chakras are associated with seven basic levels of consciousness. The seven master chakra centers are identified as:

Chakra one, the base or root chakra, is located at the base of the spine and is associated with survival. Its element is earth.

Chakra two, the sacral chakra, is located in the lower abdomen and is associated with emotions and sexuality. Its element is water.

Chakra three, the solar plexus chakra, is located in the solar plexus and is associated with personal power and metabolic energy. Its element is fire.

Chakra four, the heart chakra, is located over the sternum and is associated with love. Its element is air.

Chakra five, the throat chakra, is located in the throat and is associated with communication and creativity. Its element is sound.

Chakra six, the brow or third eye chakra, is located in the center of the forehead and is associated with clairvoyance intuition and imagination. Its element is light.

Chakra seven, the crown chakra, is located at the top of the head as is associated with knowledge and understanding. Its element is thought.

Further aromatherapy usage is applied to the general energy field produced by the actual physiological electrochemical nature of the body producing magnetic and other fields which are actually measurable with scientific measuring instruments. A great deal of medical research has been done recently with the elecromagnetic related fields of the brain which can be measured so that healthy brain activity can be differentiated from unhealthy brain activity based on the observations of the fields. An aura is considered an invisible envelope of vital energy, which apparently radiates from everything in nature: minerals, plants, animals, and humans. It is not visible to normal vision, but is measurable with scientific instruments and, it is claimed, may be seen by those who are sensitive to these emanations as a halo of light. It often appears as a multicolored mist that fades off into space with no definite boundary, and having sparks, rays, and streamers, as backed up by scientific measurement.

Prior art patents related to aroma therapy formulations and methods of delivery have varying degrees of success and generally tend to provide solutions which are much too strong for the intended purposes and also often applied topically with an oil base.

Prior art U.S. Pat. No. 6,016,450, issued Jan. 18, 2000 to Crock, provides a method and apparatus for stimulating the healing of living tissue using aura therapy. The therapeutic method and apparatus to induce the healing of tissue cells by an electrical stimulus to a tissue area with the problematic condition by the application of low density charges of alternate polarities. The apparatus performs the therapeutic function by alternately charging and discharging the cells in fifteen-minute intervals over periods of time from 1 to 12 hours.

Prior art U.S. Pat. No. 6,014,973, issued Jan. 18, 2000 to Kaufman, shows a method of healing a living body and a device are provided comprising a combination of five healing elements: a first healing element comprising moss agate, a second healing element comprising copper, a third healing element comprising malachite, a fourth healing element comprising tourmaline, and a fifth healing element comprising a skeletal material from a once-living creature. The method comprises the steps of supporting each of the healing elements in a position proximate to or in direct contact with a living body, either simultaneously or in sequence. The device for effecting this healing method comprises a securing means to which the above described healing elements are secured. The method includes placing the healing elements on the chakras of the body.

Prior art U.S. patent application 20020081341, issued Jun. 27, 2002 to Sott, claims an invention which is directed to a method system and apparatus for formulating an essential oil based composition. In formulating the composition an analysis of an individual is performed. From the analysis a value is calculated which determines whether one or more essential oils may be excluded from an essential oil universe comprising a plurality of essential oils to form an active essential oil universe. A sub-set of the essential oil universe is generated that contains only oils associated with the given value. A primary essential oil from the sub-set based on the analysis; and a primary essential oil family set is generated. A second essential oil and any subsequent oils is generated from the primary essential oil family set, based on the analysis. These steps for selecting the second essential oil may be repeated for any additional essential oils to be selected. This patent application also discusses the use of essential oils in treatment by aromatherapy of the various Chakras in a user.

Prior art U.S. Pat. No. 4,671,959, issued Jun. 9, 1987 to Warren, describes a method for reducing physiological and/or subjective reactivity to stress in humans being subjected to stress conditions. The method consists of administering to such humans an effective amount of a physiological and/or subjective stress reactivity-reducing substance selected from the group consisting of: (i) Nutmeg Oil; (ii) Mace Extract; (iii) Neroli Oil; (iv) Valerian Oil; (v) Myristicin; (vi) Isoelemicin; and (vii) Elemicin. Administration is through inhalation or transdermally using one or more of the above ingredients alone or in a suitable composition such as ethanol and/or a perfume composition, cologne or perfumed article (e.g., air freshener or deodorant stick). Also described is a method for detecting the reduction of physiological and/or subjective reactivity to stress in a human.

Prior art U.S. Pat. No. 4,670,463, issued Jun. 2, 1987 to Warren, discloses a method for reducing physiological and/or subjective reactivity to stress in humans being subjected to stress conditions. The method consists of administering to such humans an effective amount of a physiological and/or subjective stress reactivity-reducing substance selected from the group consisting of: (i) Nutmeg Oil; (ii) Mace Extract; (iii) Neroli Oil; (iv) Valerian Oil; (v) Myristicin; (vi) Isoelemicin; and (vii) Elemicin. Administration is through inhalation or transdermally using one or more of the above ingredients alone or in a suitable composition such as ethanol and/or a perfume composition, cologne or perfume article (e.g., air freshener or deodorant stick). Also described is a method for detecting the reduction of physiological and/or subjective reactivity to stress in a human.

Prior art U.S. Pat. No. 4,670,264, issued Jun. 2, 1987 to Warren, indicates a method for reducing physiological and/or subjective reactivity to stress in humans being subjected to stress conditions. The method consists of administering to such humans an effective amount of a physiological and/or subjective stress reactivity-reducing substance selected from the group consisting of: (i) Nutmeg Oil; (ii) Mace Extract; (iii) Neroli Oil; (iv) Valerian Oil; (v) Myristicin; (vi) Isoelemicin; and (vii) Elemicin. Administration is through inhalation or transdermally using one or more of the above ingredients alone or in a suitable composition such as ethanol and/or a perfume composition, cologne or perfumed article (e.g., air freshener or deodorant stick). Also described is a method for detecting the reduction of physiological and/or subjective reactivity to stress in a human.

What is needed is an aroma therapy mixture which is formulated with appropriate combinations of essential oils for specific purposes in an appropriate intensity for the intended purpose and using an appropriate distillate for mixing with the essential oils to dispense the mixture as desired.

### SUMMARY OF THE INVENTION

An object of the present invention is to provide an aromatherapy treatment system that utilizes a sprayable solution.

Another object of the present invention is to provide appropriate combinations of essential oils for application to each of the individual master chakras, or to the aura.

One more object of the present invention is to provide an appropriate intensity of the essential oil solution for the intended purpose.

An additional object of the present invention is to provide an appropriate distillate for mixing with the essential oils to dispense the mixture as desired.

A further object of the present invention is to provide a form of aromatherapy that may be used in conjunction with meditation.

A contributory object of the present invention is to place the essential oil solution within a dark bottle or container, which allows for a longer shelf life and helps to maintain the potency of the essential oils.

An added object of the present invention is to provide a method of cleansing the aura.

Yet another object of the present invention is to provide a method of protecting the aura.

An ensuing object of the present invention is to provide a method of moving energy through one or all of the seven master chakras, to help facilitate healing the physical body.

An ancillary object of the present invention is to provide a method of moving energy through one or all of the seven master chakras, to help facilitate healing the emotional body.

In brief, an aromatherapy treatment that comprises a solution of specific mixtures of essential oils and distilled water. The solution is placed within a dark bottle or container to lengthen its shelf life and preserve the potency of the essential oils. A special blend of essential oils has been designed for each of the seven master chakras and aura. The solution is applied by means of spraying on the chakra point in need of treatment, or around the perimeter of the body for treatment of the aura. The use of the individual solutions on the appropriate chakras helps move energy through them, thereby facilitating the healing process of the physical and/or the emotional body. The use of the solution created for the aura helps to cleanse and protect the aura. The treatment may be used in conjunction with meditation.

An advantage of the present invention is that it provides mixtures of essentials oils that work more effectively in unison, as opposed to independently.

Another advantage of the present invention is that the essential oils are diluted and less likely to irritate the skin.

An additional advantage of the present invention is in being easy to apply.

One more advantage of the present invention is being able to directly apply it to the chakras or the aura.

Yet another advantage of the present invention is that the essential oils are mixed in the appropriate intensity for the intended purpose.

Still another advantage of the present invention is that it absorbs more quickly than mixtures in oil based, creams.

A further advantage of the present invention is that it does not require the use of massage therapy.

### BEST MODE FOR CARRYING OUT THE INVENTION

An aromatherapy treatment system utilizes a sprayable solution of essential oils in distilled water. The system comprises a solution that has less than a three percent quantity of at least one essential oil mixed in distilled water, the solution is capable of being sprayed in the air around a body of a person using a spray means for spraying the solution in the air. The spray means is capable of spraying a desired amount of the solution around a portion of the body of the person to produce healthful effects for the person. The aromatherapy treatment helps to create healthful effects by the application of the solution containing the at least one essential oil to the correlating portion of the body. Each of the master chakra centers on the body of the person and the person's aura has at least one essential oil that is associated with it.

The crown or seventh chakra located around the top of the head of the person has a quantity of at least one essential oil associated with it. The solution for the crown or seventh chakra preferably comprises a one percent solution of frankincense, rosewood, and lavender mixed in distilled water.

The brow or sixth chakra located around the front and back of the head of the person has a quantity of at least one essential oil associated with it. The solution for the brow or sixth chakra preferably comprises a one percent solution of rosemary, juniper berry, helichrysum, and thyme mixed in distilled water.

The throat or fifth chakra located around the front and back of the throat of the person has a quantity of at least one essential oil associated with it. The solution for the throat or fifth chakra preferably comprises a one percent solution of German chamomile, Roman chamomile, and lavender mixed in distilled water.

The heart or fourth chakra located around the front and back of the sternum of the person has a quantity of at least one essential oil associated with it. The solution for the heart or fourth chakra preferably comprises a less than one percent solution of bergamot, rose, and Melissa mixed in distilled water.

The solar plexus or third chakra located around the front and back of the solar plexus of the person has a quantity of at least one essential oil associated with it. The solution for the solar plexus or third chakra preferably comprises a one percent solution of Mandarin, vetiver, juniper berry, and neroli mixed in distilled water.

The sacral or second chakra located around the front and back of the lower abdomen of the person has a quantity of at least one essential oil associated with it. The solution for the sacral or second chakra preferably comprises a one percent solution of sandalwood, jasmine, and rose mixed in distilled water.

The base, root or first chakra located around the base of the spine of the person has a quantity of at least one essential oil associated with it. The solution for the base, root or first chakra preferably comprises a one percent solution of frankincense, rosewood, and cedarwood mixed in distilled water.

The aura emanating from the body of the person has at least one essential oil associated with it. The solution for treatment of the aura preferably has a less than three percent solution of juniper berry, rosemary, and Atlas cedarwood mixed in distilled water and is sprayed around the perimeter of the body of the person.

In practice, the correlating solution is sprayed on one or more of the seven master chakras to facilitate the movement of energy through them. Chakras two through six are treated on both a front and a back of the person's body. The solution is sprayed around the perimeter of the body for treatment of the aura. The solution may be used in conjunction with meditation. The person being treated is given at least one instruction concerning how they should behave to receive optimum benefit from the solution. Preferably the person treated is given instructions on how to relax and focus energy on the chakra being treated.

It is understood that the preceding description is given merely by way of illustration and not in limitation of the invention and that various modifications may be made thereto without departing from the spirit of the invention as claimed.

What is claimed is:

1. An aroma therapy treatment system utilizing a sprayable solution of essential oils in distilled water, the system comprising:
a solution comprising less than a three percent quantity of at least one essential oil mixed in distilled water, the solution capable of being sprayed in the air around a body of a person;
a spray means for spraying the solution in the air, the spray means capable of spraying a desired amount of the solution around at least one portion of the body of the person to produce healthful effects for the person associated with an aromatherapy treatment associating healthful effects from the application of the solution of the at least one essential oil to the at least one portion of the body of the person;
at least one instruction given to the person concerning how the person should behave to receive optimum benefit from the solution.

2. The system of claim 1 wherein the at least one essential oil is associated with one of the master chakra centers on the body of the person.

3. The system of claim 2 wherein the quantity of at least one essential oil comprises a one percent solution of Frankincense, Rosewood, and Lavender mixed in distilled water and the portion of the body comprises the crown or seventh chakra around the top of the head of the person.

4. The system of claim 2 wherein the quantity of at least one essential oil comprises a one percent solution of rosemary, juniper berry, helichrysum, and thyme mixed in distilled water and the portion of the body comprises the brow or sixth chakra around the front and back of the head of the person.

5. The system of claim 2 wherein the quantity of at least one essential oil comprises a one percent solution of German chamomile, Roman chamomile, and lavender mixed in distilled water and the portion of the body comprises the throat or fifth chakra around the front and back of the throat of the person.

6. The system of claim 2 wherein the quantity of at least one essential oil comprises a less than one percent solution of bergamot, rose, and Melissa mixed in distilled water and the portion of the body comprises the heart or fourth chakra around the front and back of the sternum of the person.

7. The system of claim 2 wherein the quantity of at least one essential oil comprises a one percent solution of Mandarin, vetiver, juniper berry, and neroli mixed in distilled water and the portion of the body comprises the solar plexus or third chakra around the front and back of the solar plexus of the person.

8. The system of claim 2 wherein the quantity of at least one essential oil comprises a one percent solution of sandalwood, jasmine, and rose mixed in distilled water and the portion of the body comprises the sacral or second chakra around the front and back of the lower abdomen of the person.

9. The system of claim 2 wherein the quantity of at least one essential oil comprises a one percent solution of frankincense, rosewood, and cedarwood mixed in distilled water and the portion of the body comprises the base, root or first chakra around the front and the back of the base of the spine of the person.

10. The system of claim 1 wherein the at least one essential oil is associated with the aura of the body of the person.

11. The system of claim 10 wherein the quantity of at least one essential oil comprises a less than three percent solution of juniper berry, rosemary, and Atlas cedarwood mixed in distilled water and is sprayed around the perimeter of the body of the person.
